(19) United States
(12) Patent Application Publication
(10) Pub. No.: US 2023/0059289 A1
(43) Pub. Date: Feb. 23, 2023

(54) Drug Delivery Pump

(71) Applicant: Ticona LLC, Florence, KY (US)

(72) Inventors: Youhao Yang, Union, KY (US); Young Shin Kim, Cincinnati, OH (US)

(21) Appl. No.: 17/867,777

(22) Filed: Jul. 19, 2022

Related U.S. Application Data
(60) Provisional application No. 63/230,099, filed on Aug. 6, 2021.

(51) Int. Cl. A61L 31/06; A61M 5/158; A61K 38/28

(52) U.S. Cl. CPC A61L 31/06; A61M 5/158; A61K 38/28

(57) ABSTRACT

A medical device for administering a drug compound (e.g., insulin) to a user is provided. The medical device comprises a drug reservoir that includes the drug compound, a pump that is in fluid communication with the drug reservoir, and a housing that encloses the drug reservoir and the pump. The medical device comprises a polymer composition containing a polymer matrix that includes a liquid crystalline polymer containing repeating units derived from naphthenic hydroxycarboxylic and/or dicarboxylic acids in an amount of about 25 mol. % or less of the polymer. The polymer composition exhibits a melt viscosity of about 50 Pa-s or less.

### RELATED APPLICATION

The present application is based upon and claims priority to U.S. Provisional Patent Application Ser. No. 63/230,099, having a filing date of Aug. 6, 2022, which is incorporated herein by reference.

### BACKGROUND OF THE INVENTION

Drug delivery pumps (e.g., insulin pumps) can help a user keep a desired drug infusion level within a target range based on his or her individual needs. Many of these pumps are ambulatory or “wearable” due to their low cost and convenience to use. Such wearable drug delivery pumps are generally composed of multiple thin-walled, precise dimension components (e.g., housings). The molding of these parts with a multi-cavity mold requires a material with balanced mechanical and flow properties. Tools with more cavities requires a low melt viscosity material that can fill the mold properly without short shots. In many cases, the molder needs to increase the pressure to better fill the mold, but this increases costs and the wear on the molding equipment. As such, a need currently exists for a polymer composition that has improved properties for use in a drug delivery pump.

### SUMMARY OF THE INVENTION

In accordance with one embodiment of the present invention, a medical device for administering a drug compound to a user is disclosed. The medical device comprises a drug reservoir that includes the drug compound, a pump that is in fluid communication with the drug reservoir, and a housing that encloses the drug reservoir and the pump. The medical device comprises a polymer composition containing a polymer matrix that includes a liquid crystalline polymer containing repeating units derived from naphthenic hydroxycarboxylic and/or dicarboxylic acids in an amount of about 25 mol. % or less of the polymer. The polymer composition exhibits a melt viscosity of about 50 Pa-s or less as determined in accordance with ISO Test No. 11443:2014 at a shear rate of 1,000 seconds⁻¹ and temperature of about 30° C. above the melting temperature.

Other features and aspects of the present invention are set forth in greater detail below.

### BRIEF DESCRIPTION OF THE FIGURES

A full and enabling disclosure of the present invention, including the best mode thereof to one skilled in the art, is set forth more particularly in the remainder of the specification, including reference to the accompanying figures, in which:

FIG. 1A is a schematic view of one embodiment of a medical device of the present invention;

FIG. 1B is an illustration of one embodiment of a priming mechanism that may be employed in the medical device of FIG. 1A;

FIG. 2 is a block diagram depicting certain components of one embodiment of the medical device of the present invention; and

FIGS. 3A-3B illustrate exemplary injection assemblies for use in one embodiment of the medical device of the present invention.

### DETAILED DESCRIPTION

It is to be understood by one of ordinary skill in the art that the present discussion is a description of exemplary embodiments only, and is not intended as limiting the broader aspects of the present invention.

Generally speaking, the present invention is directed to a medical device for administering a drug compound to a user (e.g., human, pet, farm animal, racehorse, etc.) over an extended period of time to help prohibit and/or treat a condition, disease, and/or cosmetic state of the patient. The device contains a housing within which a drug reservoir is disposed that includes the drug compound and a pump that is fluid communication with the drug reservoir. At least a portion of the device includes a polymer composition containing a liquid crystalline polymer. Through careful control over the specific nature and concentration of the components employed in the composition (e.g., liquid crystalline polymer), the present inventors have discovered that the resulting composition can exhibit a melt viscosity that is sufficiently low to enable it to be readily molded into the small dimensions required for a medical device. For example, the polymer composition may have a melt viscosity of about 50 Pa-s or less, in some embodiments about 48 Pa-s or less, in some embodiments from about 1 Pa-s to about 45 Pa-s, and in some embodiments, from about 2 to about 42 Pa-s, as determined in accordance with ISO Test No. 11443:2014 at a shear rate at a shear rate of 1,000 seconds⁻¹ at a temperature of about 30° C. above the melting temperature (e.g., about 350° C.).

Conventionally, it was believed that polymer compositions exhibiting such a low melt viscosity would not also possess sufficiently good thermal and mechanical properties to enable good physical integrity for use in forming medical device components having a consistent shape and size. Contrary to conventional thought, however, the resulting polymer composition can also possess both excellent thermal and mechanical properties. For example, the polymer composition may have a melting temperature of about 285° C. or more, in some embodiments from about 290° C. to about 350° C., and in some embodiments, from about 300° C. to about 330° C., such as determined in accordance with ISO 11357-2:2020. Even at such melting temperatures, the ratio of the deflection temperature under load (“DTUL”), a measure of short-term heat resistance, to the melting temperature may still remain relatively high, which can, among other things, allow the use of high-speed processes for forming the medical device. For example, the ratio may range from about 0.5 to about 1.00, in some embodiments from about 0.65 to about 0.95, and in some embodiments from about 0.75 to about 0.85. The specific DTUL values may, for instance, be about 160° C. or more, in some embodiments from about 200° C. to about 350° C., in some embodiments from about 230° C. to about 320° C., and in some embodiments from about 235° C. to about 300° C., such as determined in accordance with ISO Test No. 75-2:2013 at a load of 1.8 Megapascals.

The polymer composition may also be generally stiff in nature so that it is capable of maintaining the desired degree of physical integrity during formation of the medical device. Such stiffness may be generally characterized by a low tensile elongation and/or a high tensile modulus. For example, the tensile elongation may be about 5% or less, in some embodiments about 4% or less, in some embodiments, from about 0.1 to about 3.5%, in some embodiments from about 0.2% to about 3%, and in some embodiments, from about 0.5% to about 2.5%, such as determined in accordance with ISO Test No. 527:2019 at a temperature of about 23° C. The tensile modulus may likewise be about 7,000 MPa or more, in some embodiments about 7,500 MPa or more, in some embodiments from about 8,000 MPa to about 25,000 MPa, in some embodiments about 8,500 MPa to about 20,000 MPa, and in some embodiments from about 9,000 MPa to about 15,000 MPa, such as determined in accordance with ISO Test No. 527:2019 at a temperature of about 23° C. The polymer composition may also exhibit other good mechanical properties. For example, the polymer composition may exhibit a tensile strength of about 100 MPa or more, in some embodiments about 120 MPa or more, and in some embodiments from about 140 MPa to about 250 MPa, such as determined in accordance with ISO Test No. 527:2019 at a temperature of about 23° C. The polymer composition may also exhibit a flexural strength of from about 100 to about 500 MPa, in some embodiments from about 150 to about 300 MPa, and in some embodiments, from about 180 to about 240 MPa and/or flexural modulus of from about 5,000 MPa to about 20,000 MPa, in some embodiments, from about 8,000 MPa to about 18,000 MPa, and in some embodiments, from about 10,000 MPa to about 15,000 MPa. The flexural properties may be determined in accordance with ISO Test No. 178:2019 (technically equivalent to ASTM D790-10) at 23° C. The composition may also exhibit a Charpy unnotched and/or notched impact strength of about 30 kJ/m² or more, in some embodiments from about 40 to about 80 kJ/m², and in some embodiments, from about 50 to about 70 kJ/m², measured at 23° C. according to ISO Test No. 179-1:2010.

Various embodiments of the present invention will now be described in more detail.

I. Polymer Composition

A. Polymer Matrix

The polymer matrix contains one or more liquid crystalline polymers, which are generally classified as “thermotropic” to the extent that they can possess a rod-like structure and exhibit a crystalline behavior in their molten state (e.g., thermotropic nematic state). The polymers have a relatively high melting temperature, such as about 285° C. or more, in some embodiments from about 290° C. to about 350° C., and in some embodiments, from about 300° C. to about 330° C. Such polymers may be formed from one or more types of repeating units as is known in the art. A liquid crystalline polymer may, for example, contain one or more aromatic ester repeating units generally represented by the following Formula (I):

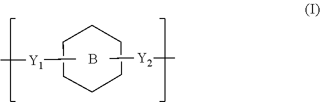

wherein,

ring B is a substituted or unsubstituted 6-membered aryl group (e.g., 1,4-phenylene or 1,3-phenylene), a substituted or unsubstituted 6-membered aryl group fused to a substituted or unsubstituted 5- or 6-membered aryl group (e.g., 2,6-naphthalene), or a substituted or unsubstituted 6-membered aryl group linked to a substituted or unsubstituted 5- or 6-membered aryl group (e.g., 4,4-biphenylene); and

Y₁ and Y₂ are independently O, C(O), NH, C(O)HN, or NHC(O).

Typically, at least one of Y₁ and Y₂ are C(O). Examples of such aromatic ester repeating units may include, for instance, aromatic dicarboxylic repeating units (Y₁ and Y₂ in Formula I are C(O)), aromatic hydroxycarboxylic repeating units (Y₁ is O and Y₂ is C(O) in Formula I), as well as various combinations thereof.

Aromatic hydroxycarboxylic repeating units, for instance, may be employed that are derived from aromatic hydroxycarboxylic acids, such as, 4-hydroxybenzoic acid; 4-hydroxy-4′-biphenylcarboxylic acid; 2-hydroxy-6-naphthoic acid; 2-hydroxy-5-naphthoic acid; 3-hydroxy-2-naphthoic acid; 2-hydroxy-3-naphthoic acid; 4′-hydroxyphenyl-4-benzoic acid; 3′-hydroxyphenyl-4-benzoic acid; 4′-hydroxyphenyl-3-benzoic acid, etc., as well as alkyl, alkoxy, aryl and halogen substituents thereof, and combination thereof. Particularly suitable aromatic hydroxycarboxylic acids are 4-hydroxybenzoic acid (“HBA”) and 6-hydroxy-2-naphthoic acid (“HNA”). To help achieve the desired properties, the repeating units derived from hydroxycarboxylic acids (e.g., HBA and/or HNA) typically constitute about 50 mol. % or more, in some embodiments about 60 mol. % or more, in some embodiments about 70 mol. % or more, in some embodiments about 80 mol. % or more, in some embodiments from about 85 mol. % to 100 mol. %, and in some embodiments, from about 90 mol. % to about 99 mol. % of the polymer.

Aromatic dicarboxylic repeating units may also be employed that are derived from aromatic dicarboxylic acids, such as terephthalic acid, isophthalic acid, 2,6-naphthalenedicarboxylic acid, diphenyl ether-4,4′-dicarboxylic acid, 1,6-naphthalenedicarboxylic acid, 2,7-naphthalenedicarboxylic acid, 4,4′-dicarboxybiphenyl, bis(4-carboxyphenyl)ether, bis(4-carboxyphenyl)butane, bis(4-carboxyphenyl)ethane, bis(3-carboxyphenyl)ether, bis(3-carboxyphenyl)ethane, etc., as well as alkyl, alkoxy, aryl and halogen substituents thereof, and combinations thereof. Particularly suitable aromatic dicarboxylic acids may include, for instance, terephthalic acid (“TA”), isophthalic acid (“IA”), and 2,6-naphthalenedicarboxylic acid (“NDA”). When employed, repeating units derived from aromatic dicarboxylic acids (e.g., IA, TA, and/or NDA) may each optionally constitute from about 0.1 mol. % to about 20 mol. %, in some embodiments from about 0.5 mol. % to about 15 mol. %, and in some embodiments, from about 1 mol. % to about 10% of the polymer.

Other repeating units may also be employed in the polymer. In certain embodiments, for instance, repeating units may be employed that are derived from aromatic diols, such as hydroquinone, resorcinol, 2,6-dihydroxynaphthalene, 2,7-dihydroxynaphthalene, 1,6-dihydroxynaphthalene, 4,4′-dihydroxybiphenyl (or 4,4′-biphenol), 3,3′-dihydroxybiphenyl, 3,4′-dihydroxybiphenyl, 4,4′-dihydroxybiphenyl ether, bis(4-hydroxyphenyl)ethane, etc., as well as alkyl, alkoxy, aryl and halogen substituents thereof, and combinations thereof. Particularly suitable aromatic diols may include, for instance, hydroquinone (“HQ”) and 4,4′-biphenol (“BP”). When employed, repeating units derived from aromatic diols (e.g., HQ and/or BP) may each optionally constitute from about 0.1 mol. % to about 20 mol. %, in some embodiments from about 0.5 mol. % to about 15 mol. %, and in some embodiments, from about 1 mol. % to about 10% of the polymer.

Repeating units may also be employed, such as those derived from aromatic amides (e.g., acetaminophen (“APAP”)) and/or aromatic amines (e.g., 4-aminophenol (“AP”), 3-aminophenol, 1,4-phenylenediamine, 1,3-phenylenediamine, etc.). When employed, repeating units derived from aromatic amides (e.g., APAP) and/or aromatic amines (e.g., AP) may optionally constitute from about 0.1 mol. % to about 15 mol. %, in some embodiments from about 0.5 mol. % to about 10 mol. %, and in some embodiments, from about 1 mol. % to about 6% of the polymer. It should also be understood that various other monomeric repeating units may be incorporated into the polymer. For instance, in certain embodiments, the polymer may contain one or more repeating units derived from non-aromatic monomers, such as aliphatic or cycloaliphatic hydroxycarboxylic acids, dicarboxylic acids, diols, amides, amines, etc. Of course, in other embodiments, the polymer may be “wholly aromatic” in that it lacks repeating units derived from non-aromatic (e.g., aliphatic or cycloaliphatic) monomers.

In certain embodiments, the liquid crystalline polymer may be a “low naphthenic” polymer to the extent that it contains a relatively low content of repeating units derived from naphthenic hydroxycarboxylic acids and naphthenic dicarboxylic acids, such as NDA, HNA, or combinations thereof. That is, the total amount of repeating units derived from naphthenic hydroxycarboxylic and/or dicarboxylic acids (e.g., NDA, HNA, or a combination of HNA and NDA) is typically about 25 mol. % or less, in some embodiments from about 10 mol. % to about 25 mol. %, in some embodiments from about 12 mol. % to about 24 mol. %, and in some embodiments, from about 15 mol. % to about 23 mol. % of the polymer. The liquid crystalline polymer may also contain various other monomers. For example, the polymer may also contain repeating units derived from HBA, such as in an amount of from about 75 mol. % to about 90 mol. %, and in some embodiments from about 76 mol. % to about 88 mol. %, and in some embodiments, from about 77 mol. % to about 85 mol. %. When employed, the molar ratio of HBA to HNA, for example, may be selectively controlled within a specific range to help achieve the desired properties, such as from about 0.5 to about 20, in some embodiments from about 1 to about 10, in some embodiments from about 2 to about 8, and in some embodiments, from about 3 to about 6. The polymer may also contain aromatic dicarboxylic acid(s) (e.g., IA and/or TA) in an amount of from about 0.1 mol. % to about 20 mol. %; and/or aromatic diol(s) (e.g., BP and/or HQ) in an amount of from about 0.2 mol. % to about 10 mol. %, and in some embodiments, from about 0.5 mol. % to about 5 mol. %. In some cases, however, it may be desired to minimize the presence of such monomers in the polymer to help achieve the desired properties. For example, the total amount of aromatic dicarboxylic acid(s) (e.g., IA and/or TA) may be about 20 mol % or less, in some embodiments about 15 mol. % or less, in some embodiments about 10 mol. % or less, in some embodiments, from 0 mol. % to about 5 mol. %, and in some embodiments, from 0 mol. % to about 2 mol. % of the polymer. Although not required in all instances, it is often desired that a substantial portion of the polymer matrix is formed from such low naphthenic polymers. For example, low naphthenic polymers such as described herein typically constitute 50 wt. % or more, in some embodiments about 65 wt. % or more, in some embodiments from about 70 wt. % to 100 wt. %, and in some embodiments, from about 80 wt. % to 100% of the polymer matrix (e.g., 100 wt. %).

Regardless of the particular constituents and nature of the polymer, the liquid crystalline polymer may be prepared by initially introducing the aromatic monomer(s) used to form the ester repeating units (e.g., aromatic hydroxycarboxylic acid, aromatic dicarboxylic acid, etc.) and/or other repeating units (e.g., aromatic diol, aromatic amide, aromatic amine, etc.) into a reactor vessel to initiate a polycondensation reaction. The particular conditions and steps employed in such reactions are well known, and may be described in more detail in U.S. Pat. No. 4,161,470 to Calundann; U.S. Pat. No. 5,616,680 to Linstid, III, et al.; U.S. Pat. No. 6,114,492 to Linstid, III, et al.; U.S. Pat. No. 6,514,611 to Shepherd, et al.; and WO 2004/058851 to Waggoner. The vessel employed for the reaction is not especially limited, although it is typically desired to employ one that is commonly used in reactions of high viscosity fluids. Examples of such a reaction vessel may include a stirring tank-type apparatus that has an agitator with a variably-shaped stirring blade, such as an anchor type, multistage type, spiral-ribbon type, screw shaft type, etc., or a modified shape thereof. Further examples of such a reaction vessel may include a mixing apparatus commonly used in resin kneading, such as a kneader, a roll mill, a Banbury mixer, etc.

If desired, the reaction may proceed through the acetylation of the monomers as known the art. This may be accomplished by adding an acetylating agent (e.g., acetic anhydride) to the monomers. Acetylation is generally initiated at temperatures of about 90° C. During the initial stage of the acetylation, reflux may be employed to maintain vapor phase temperature below the point at which acetic acid byproduct and anhydride begin to distill. Temperatures during acetylation typically range from between 90° C. to 150° C., and in some embodiments, from about 110° C. to about 150° C. If reflux is used, the vapor phase temperature typically exceeds the boiling point of acetic acid, but remains low enough to retain residual acetic anhydride. For example, acetic anhydride vaporizes at temperatures of about 140° C. Thus, providing the reactor with a vapor phase reflux at a temperature of from about 110° C. to about 130° C. is particularly desirable. To ensure substantially complete reaction, an excess amount of acetic anhydride may be employed. The amount of excess anhydride will vary depending upon the particular acetylation conditions employed, including the presence or absence of reflux. The use of an excess of from about 1 to about 10 mole percent of acetic anhydride, based on the total moles of reactant hydroxyl groups present is not uncommon.

Acetylation may occur in in a separate reactor vessel, or it may occur in situ within the polymerization reactor vessel. When separate reactor vessels are employed, one or more of the monomers may be introduced to the acetylation reactor and subsequently transferred to the polymerization reactor. Likewise, one or more of the monomers may also be directly introduced to the reactor vessel without undergoing pre-acetylation.

In addition to the monomers and optional acetylating agents, other components may also be included within the reaction mixture to help facilitate polymerization. For instance, a catalyst may be optionally employed, such as metal salt catalysts (e.g., magnesium acetate, tin(I) acetate, tetrabutyl titanate, lead acetate, sodium acetate, potassium acetate, etc.) and organic compound catalysts (e.g., N-methylimidazole). Such catalysts are typically used in amounts of from about 50 to about 500 parts per million based on the total weight of the recurring unit precursors. When separate reactors are employed, it is typically desired to apply the catalyst to the acetylation reactor rather than the polymerization reactor, although this is by no means a requirement.

The reaction mixture is generally heated to an elevated temperature within the polymerization reactor vessel to initiate melt polycondensation of the reactants. Polycondensation may occur, for instance, within a temperature range of from about 250° C. to about 380° C., and in some embodiments, from about 280° C. to about 380° C. For instance, one suitable technique for forming the aromatic polyester may include charging precursor monomers and acetic anhydride into the reactor, heating the mixture to a temperature of from about 90° C. to about 150° C. to acetylize a hydroxyl group of the monomers (e.g., forming acetoxy), and then increasing the temperature to from about 280° C. to about 380° C. to carry out melt polycondensation. As the final polymerization temperatures are approached, volatile byproducts of the reaction (e.g., acetic acid) may also be removed so that the desired molecular weight may be readily achieved. The reaction mixture is generally subjected to agitation during polymerization to ensure good heat and mass transfer, and in turn, good material homogeneity. The rotational velocity of the agitator may vary during the course of the reaction, but typically ranges from about 10 to about 100 revolutions per minute (“rpm”), and in some embodiments, from about 20 to about 80 rpm. To build molecular weight in the melt, the polymerization reaction may also be conducted under vacuum, the application of which facilitates the removal of volatiles formed during the final stages of polycondensation. The vacuum may be created by the application of a suctional pressure, such as within the range of from about 5 to about 30 pounds per square inch (“psi”), and in some embodiments, from about 10 to about 20 psi.

Following melt polymerization, the molten polymer may be discharged from the reactor, typically through an extrusion orifice fitted with a die of desired configuration, cooled, and collected. Commonly, the melt is discharged through a perforated die to form strands that are taken up in a water bath, pelletized and dried. In some embodiments, the melt polymerized polymer may also be subjected to a subsequent solid-state polymerization method to further increase its molecular weight. Solid-state polymerization may be conducted in the presence of a gas (e.g., air, inert gas, etc.). Suitable inert gases may include, for instance, include nitrogen, helium, argon, neon, krypton, xenon, etc., as well as combinations thereof. The solid-state polymerization reactor vessel can be of virtually any design that will allow the polymer to be maintained at the desired solid-state polymerization temperature for the desired residence time. Examples of such vessels can be those that have a fixed bed, static bed, moving bed, fluidized bed, etc. The temperature at which solid-state polymerization is performed may vary, but is typically within a range of from about 250° C. to about 350° C. The polymerization time will of course vary based on the temperature and target molecular weight. In most cases, however, the solid-state polymerization time will be from about 2 to about 12 hours, and in some embodiments, from about 4 to about 10 hours.

B. Other Additives

In some cases, the polymer matrix may constitute the entire polymer composition (e.g., 100 wt. %). Nevertheless, it may be desirable in certain embodiments to include one or more additives within the polymer composition to help achieve the target properties. In such embodiments, the polymer matrix may constitute from about 50 wt. % to about 98 wt. %, in some embodiments from about 60 wt. % to about 96 wt. %, and in some embodiments, from about 75 wt. % to about 95 wt. % of the composition, as well as one or more additives (e.g., fibrous filler) in an amount of from about 2 wt. % to about 50 wt. %, in some embodiments from about 4 wt. % to about 40 wt. %, and in some embodiments, from about 5 wt. % to about 25 wt. % of the composition.

In one embodiment, for instance, a fibrous filler may be employed in the polymer composition to improve the thermal and mechanical properties of the composition without having a significant impact on flowability. The fibrous filler typically includes fibers having a high degree of tensile strength relative to their mass. For example, the ultimate tensile strength of the fibers (such as determined in accordance with ASTM D3822) is typically from about 1,000 to about 15,000 MPa, in some embodiments from about 2,000 MPa to about 10,000 MPa, and in some embodiments, from about 3,000 MPa to about 6,000 MPa. Such high strength fibers may be formed from materials that are generally insulative in nature, such as glass, ceramics (e.g., alumina or silica), aramids (e.g., Kevlar0), polyolefins, polyesters, titanium acids (e.g., titanium acid potassium available from TISMO), etc. Glass fibers are particularly suitable, such as E-glass, A-glass, C-glass, O-glass, AR-glass, R-glass, S1-glass, S2-glass, etc. Further, fibers having a certain length and diameter can help improve the mechanical properties of the resulting thermoplastic composition. For instance, particularly suitable fibers may have an average nominal length (prior to compounding) of from about 0.3 to about 10 millimeters, in some embodiments from about 0.5 to about 6 millimeters, in some embodiments, and in some embodiments, from about 1 to about 5 millimeters. The fibers may likewise have an average nominal diameter of about 5 to about 35 micrometers, in some embodiments from about 6 to about 25 micrometers, and in some embodiments, from about 7 to about 15 micrometers. Fiber length and/or diameter may be measured using a variety of known techniques, such as by scanning electronic microscopy (“SEM”).

Other additives may also be employed. For example, a mineral filler, which may be in the form of particles (e.g., platelet-shaped, flake-shaped, etc.), fibers, and so forth, may be employed. In one embodiment, for example, the mineral filler may include a particulate mineral filler, such as talc, halloysite, kaolinite, illite, montmorillonite, vermiculite, palygorskite, pyrophyllite, mica, diatomaceous earth, etc., as well as combinations thereof. Mica and/or talc may be particularly suitable. In one particular embodiment, for example, the particulate mineral filler (e.g., talc) may have a median size (e.g., D50 size) of about 10 micrometers or less, in some embodiments from about 0.1 to about 8 micrometers, in some embodiments from about 0.5 to about 5 micrometers, and in some embodiments, from about 0.6 to about 2.5 micrometers. Besides silicates, other suitable mineral filler particles may include carbonates, such as calcium carbonate (CaCO₃) or a copper carbonate hydroxide (Cu₂CO₃(OH)₂), fluorides, such as calcium fluoride (CaFl₂); phosphates, such as calcium pyrophosphate (Ca₂P₂O₇), anhydrous dicalcium phosphate (CaHPO₄), or hydrated aluminum phosphate (AlPO₄.2H₂O), glass (e.g., glass powder); etc. Mineral fibers (also known as “whiskers”) may also be employed as a mineral filler in the polymer composition. Examples of such mineral fibers include those that are derived from silicates, such as neosilicates, sorosilicates, inosilicates (e.g., calcium inosilicates, such as wollastonite; calcium magnesium inosilicates, such as tremolite; calcium magnesium iron inosilicates, such as actinolite; magnesium iron inosilicates, such as anthophyllite; etc.), phyllosilicates (e.g., aluminum phyllosilicates, such as palygorskite), tectosilicates, etc.; sulfates, such as calcium sulfates (e.g., dehydrated or anhydrous gypsum); mineral wools (e.g., rock or slag wool); glass; and so forth. Particularly suitable are inosilicates, such as wollastonite fibers available from Nyco Minerals under the trade designation NYGLOS® (e.g., NYGLOS® 4W, NYGLOS® 5, or NYGLOS® 8). In addition to possessing the size characteristics noted above, the mineral fibers may also have a relatively high aspect ratio (average length divided by median width) to help further improve the mechanical properties. For example, the mineral fibers may have an aspect ratio of from about 1 to about 50, in some embodiments from about 2 to about 20, and in some embodiments, from about 4 to about 15. The volume average length of such mineral fibers may, for example, range from about 1 to about 200 micrometers, in some embodiments from about 2 to about 150 micrometers, in some embodiments from about 5 to about 100 micrometers, and in some embodiments, from about 10 to about 50 micrometers.

A wide variety of other additional additives can also be included in the polymer composition, such as lubricants, thermally conductive fillers, pigments, antioxidants, stabilizers, surfactants, waxes, flame retardants, anti-drip additives, nucleating agents (e.g., boron nitride), flow modifiers, coupling agents, antimicrobials, pigments or other colorants, impact modifiers, and other materials added to enhance properties and processability.

II. Formation

The components used to form the polymer composition may be combined together using any of a variety of different techniques as is known in the art. In one particular embodiment, for example, the liquid crystalline polymer and other optional additives are melt processed as a mixture within an extruder to form the polymer composition. The mixture may be melt-kneaded in a single-screw or multi-screw extruder at a temperature of from about 200° C. to about 450° C. In one embodiment, the mixture may be melt processed in an extruder that includes multiple temperature zones. The temperature of individual zones is typically set within about −60° C. to about 25° C. relative to the melting temperature of the polymer. By way of example, the mixture may be melt processed using a twin screw extruder such as a Leistritz 18-mm co-rotating fully intermeshing twin screw extruder. A general purpose screw design can be used to melt process the mixture. In one embodiment, the mixture including all of the components may be fed to the feed throat in the first barrel by means of a volumetric feeder. In another embodiment, different components may be added at different addition points in the extruder, as is known. For example, the polymer may be applied at the feed throat, and certain additives (e.g., fibrous filler) may be supplied at the same or different temperature zone located downstream therefrom. Regardless, the resulting mixture can be melted and mixed then extruded through a die. The extruded polymer composition can then be quenched in a water bath to solidify and granulated in a pelletizer followed by drying.

III. Medical Device

The medical device includes one or more components that contain the polymer composition of the present invention. Any of a variety of techniques may be employed to form such components, such as embossing (e.g., hot embossing, roll-to-roll molding, etc.); molding, such as micro-molding, injection molding (e.g., low-pressure injection molding, gas injection molding, foam injection molding, etc.), compression molding (e.g., extrusion compression molding), extrusion molding; printing (e.g., three-dimensional printing); and so forth. For example, an injection molding system may be employed that includes a mold within which the polymer composition may be injected. The time inside the injector may be controlled and optimized so that polymer matrix is not pre-solidified. When the cycle time is reached and the barrel is full for discharge, a piston may be used to inject the composition to the mold cavity. Compression molding systems may also be employed. As with injection molding, the shaping of the polymer composition into the desired article also occurs within a mold. The composition may be placed into the compression mold using any known technique, such as by being picked up by an automated robot arm. The temperature of the mold may be maintained at or above the solidification temperature of the polymer matrix for a desired time period to allow for solidification. The molded product may then be solidified by bringing it to a temperature below that of the melting temperature. The resulting product may be de-molded. The cycle time for each molding process may be adjusted to suit the polymer matrix, to achieve sufficient bonding, and to enhance overall process productivity.

Generally speaking, the medical device includes one or more drug reservoirs that include at least one drug compound, a pump that is in fluid communication with the drug reservoir(s) and that is capable of receiving the drug compound from the reservoir(s) and delivering it an injection assembly. The injection assembly is in fluid communication with the pump and includes one or more components (e.g., needle, cannula, etc.) that are configured to receive the drug compound from the pump and deliver it to a user at one or more infusion sites. The drug compound may, for example, be continuously infused into the user. A housing may enclose one or more of the components noted above (e.g., reservoir(s), pump, injection assembly, etc.) to protect them from the exterior environment. Notably, one or more of the aforementioned components may be formed from the polymer composition of the present invention.

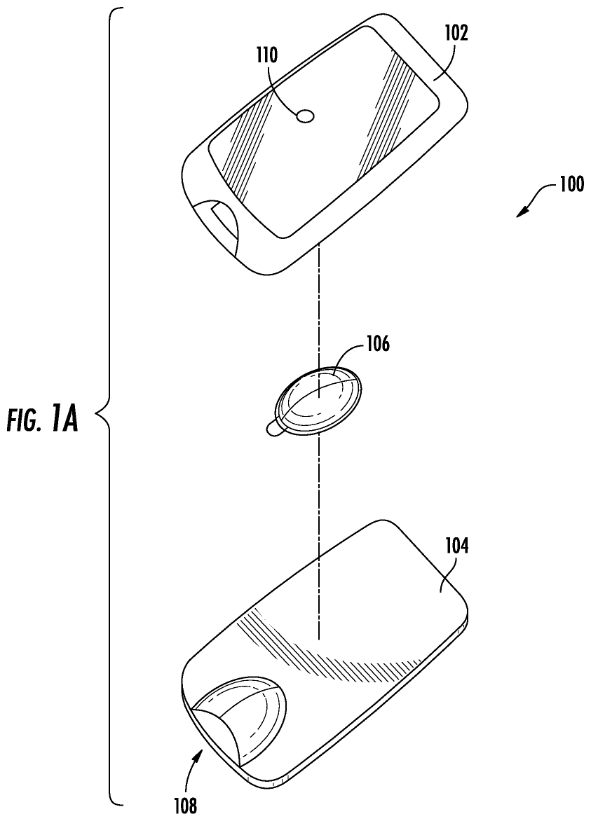

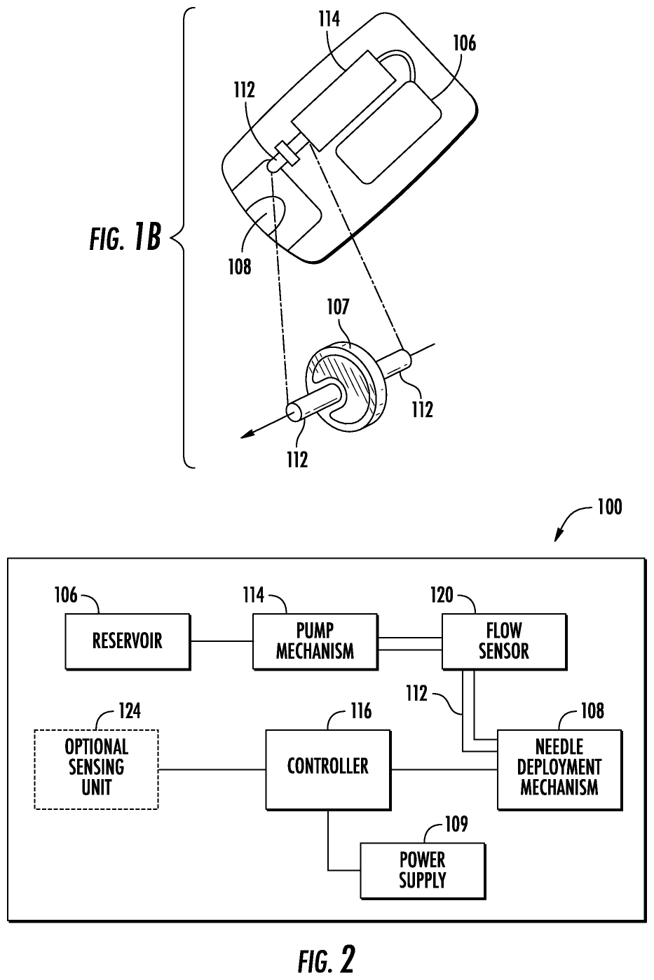

Referring to FIGS. 1A, 1B, and 2, for example, one particular embodiment of a medical device 100 is shown that is capable of delivering a liquid or gel drug compound (e.g., insulin) by continuous infusion into or through the skin of the patient. Such known medical devices are commonly referred to as “wearable patch pumps” due to their nature of being worn or affixed to the skin of a user. For instance, the medical device 100 may include a housing that is formed from an upper housing portion 102 and a lower housing portion 104, a drug reservoir 106, an injection assembly 108, and a pump 114 for controlling the delivery of the drug compound into the user's body through the injection assembly 108. The medical device 100 may also contain a microprocessor or controller 116 for directing the injection assembly 108 and pump 114, as well as monitoring and/or controlling other preferred operations and systems of the medical device 100. The medical device 100 may likewise include a flow sensor 120 and/or power supply 109, such as a battery, capacitor, or other energy harvesting system. As noted above, any of the components of the medical device 100 may contain the polymer composition of the present invention, such as the housing (e.g., housing portions 102 and/or 106), drug reservoir 106, injection assembly 108, pump 114, etc. For example, the drug reservoir 106 is generally capable of housing a volume of drug that provides an adequate supply for the extended duration of use, such as 5-7 days. The drug reservoir 106 may be provided as a prefilled reservoir packaged in the medical device 100 and may be formed from a polymeric material, such as a thermoplastic olefin polymer (e.g., cyclic olefin) and/or the polymer composition of the present invention.

Another feature that may be provided in a medical device 100 as illustrated in FIG. 1B, is directed to an automatic or semi-automatic priming of the medical device prior to use. A drug flow channel 112 may be provided within the medical device 100 that begins at the drug reservoir 106 and terminates at an infusion needle inserted by the injection assembly 108. If desired, a membrane 107 may be placed in the flow channel 112 near the injection assembly 108 to allow any air trapped in the void volume of flow channel 112 to be purged from the flow channel when the pumping 114 is initially activated. The flow of fluid in the flow channel 112 will drive the air from the flow channel 112 through the membrane 107, which inhibits the flow of fluid from the flow channel 112. If desired, the membrane 107 may also be formed from the polymer composition of the present invention.

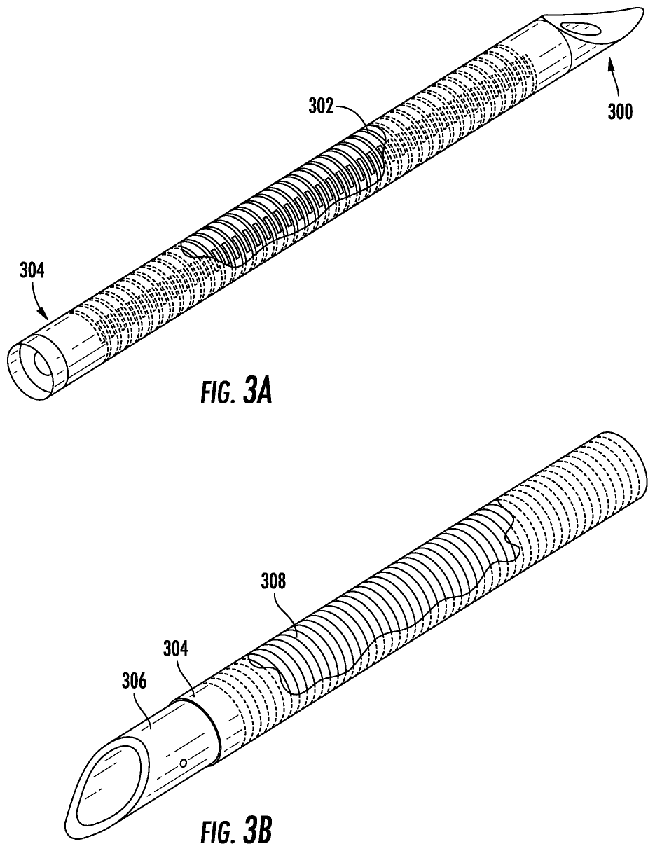

The injection assembly of the medical device may be formed using one or more insertion needles and/or cannulas as is known in the art. Referring to FIG. 3A, one particular embodiment of a cannula 300 is shown that may be employed in the injection assembly 108. In this embodiment, the cannula 300 has a sharpened tip and alternating slots 302, laser cut or chemically etched, along the shaft of the cannula. The alternating slots 302 enable the cannula to flex, yet provide a rigidity or column strength necessary for insertion into the user's skin. The cannula 300 is typically a unitary body that may be formed from a metal (e.g., stainless steel) or other materials, such as the polymer composition of the present invention. The cannula 300 may also be sheathed or coated by a sleeve 304 that provides a biocompatible outer fluid seal for enabling a drug fluid to enter to the user through the tip of the cannula. The sleeve 304 may be formed from a fluid-resistant polymeric material, such as the polymer composition of the present invention. Another exemplary embodiment of the cannula is shown in FIG. 3B in which a sharpened needle tip 306 is attached to a torsion spring 308. The torsion spring 308 provides similar benefits as the embodiment discussed in FIG. 3A and similarly also includes a sleeve 304 for sealing the fluid within the inner cavity of the torsion spring. The torsion spring 308 and cannula 300 may be provided with any suitable cross section, and may alternatively comprise a rectangular cross-section to maximize the internal diameter, as would be appreciated by one of ordinary skill in the art. Additionally, the tips of the infusion needles shown in FIGS. 3A and 3B, do not need to comprise an opening for the flow of drug to the user. It may be desirable to implement an infusion needle with a closed end, having side ports located near the tip for enabling the flow of drug to the user.

Regardless of its particular configuration, the injection assembly 108 (FIG. 1A) may contain a manual or automatic mechanism for inserting and retracting the infusion needle 122 into the user's skin. Additionally, the injection assembly 108 may be either manually or automatically actuated to insert the infusion needle into the users skin. A controller 116 may automatically actuate the injection assembly 108 after initialization of the medical device or based on some other programmed or sensed condition. Further, automatic deployment may be effected via an appropriate command received from a BGM, PDM or a host device.

One particular embodiment of the medical device 100 is a pre-programmed patch pump. Pre-programmed patch pumps may include simple intelligence for providing a customized basal infusion rate that can be varied throughout the day to match sleeping and waking insulin requirements. The pre-programmed patch pump can be programmed to deliver a drug or drugs to the user at different rates for different times of day or under different conditions. Varying drug delivery rates over time are referred to herein as a drug delivery profile. The pre-programmed patch pump can be programmed either by the manufacturing facility or a health care provider and generally requires no additional user programming. A pre-programmed patch pump may even be configured to provide multiple daily infusions and may be designed with a mechanism to enable manual actuation of an incremental bolus dose. One form of manual actuation would require the closure of an electrical contact, such as a momentary switch or two momentary switches, for an extended duration, after which a vibratory or audible signal may confirm completion of drug delivery. The pre-programmed patch pump for use in exemplary embodiments of the present invention comprises enough intelligence to perform sensing of blockage of insulin flow, a low-level of insulin in the reservoir and other fault conditions. A pre-programmed patch pump also preferably provides alarms to the user in each of these fault conditions. Pre-programmed patch pumps perform similar functions as a “smart” patch pump except for communication with a host device, thus greatly reducing the cost of providing drug therapy with such a device and enhancing the ease of use for such a device. Exemplary embodiments of medical device 100 in the present invention are preferably directed to a pre-programmable patch pump, as discussed above.

In other embodiments, the medical device 100 may also be provided as a fully-programmable (“smart”), or (“simple”) package, as would be appreciated by one of ordinary skill in the art. A fully programmable package provides the user with the greatest precision and flexibility in controlling the rate of administering a drug that is suitable for the user's lifestyle, but does require additional cost. Fully-programmable “smart” patch pumps are generally used in conjunction with a Blood Glucose Monitor (BGM) or Continuous Glucose Monitor (CGM) and a host device, such as a Personal Diabetes Monitor (PDM), to provide, through closed-loop control and sensing, a personalized basal infusion rate and bolus injections that may be activated or adjusted at any time throughout the day. “Smart” patch pumps are typically configured to be in communication with the host device, such as via a personal area network. “Smart” patch pumps may even communicate, continuously or intermittently, with the host device via a wired or other direct connection. “Simple” patch pumps can be provided with minimal or no system intelligence and generally comprise mostly mechanical systems for providing basic control of insulin infusion through either a preset basal rate or manually activated bolus injections. Each patch pump is particularly effective and desired for a certain type of user. A user's lifestyle, medical condition, financial situation and aptitude for operating a medical device largely determine which package of patch pump is suitable for that user. The specific features and functionality of exemplary embodiments of the present invention, to follow, may be implemented in each of the patch pump packages described above.

There is no particular limitation to the drug compounds that may be delivered using the medical device of the present invention. Suitable compounds may include, for instance, proteinaceous compounds, such as insulin, immunoglobulins (e.g., IgG, IgM, IgA, IgE), TNF-α, antiviral medications, etc.; polynucleotide agents, such as plasmids, siRNA, RNAi, nucleoside anticancer drugs, vaccines, etc.; small molecule agents, such as alkaloids, glycosides, phenols, etc.; anti-infection agents, hormones, drugs regulating cardiac action or blood flow, pain control; vaccines; and so forth. A non-limiting listing of agents includes anti-Angiogenesis agents, anti-depressants, antidiabetic agents, antihistamines, anti-inflammatory agents, butorphanol, calcitonin and analogs, COX-II inhibitors, dermatological agents, dopamine agonists and antagonists, enkephalins and other opioid peptides, epidermal growth factors, erythropoietin and analogs, follicle stimulating hormone, glucagon, growth hormone and analogs (including growth hormone releasing hormone), growth hormone antagonists, heparin, hirudin and hirudin analogs such as hirulog, IgE suppressors and other protein inhibitors, immunosuppressives, insulin, insulinotropin and analogs, interferons, interleukins, leutenizing hormone, leutenizing hormone releasing hormone and analogs, monoclonal or polyclonal antibodies, motion sickness preparations, muscle relaxants, narcotic analgesics, nicotine, non-steroid anti-inflammatory agents, oligosaccharides, parathyroid hormone and analogs, parathyroid hormone antagonists, prostaglandin antagonists, prostaglandins, scopolamine, sedatives, serotonin agonists and antagonists, sexual hypofunction, tissue plasminogen activators, tranquilizers, vaccines with or without carriers/adjuvants, vasodilators, major diagnostics such as tuberculin and other hypersensitivity agents. The medical device may be particularly beneficial in delivering high molecular weight drug compounds. The term “high molecular weight” generally refers to compounds having a molecular weight of about 1 kiliDalton (“kDa”) or more, in some embodiments about 2 kDa or more, and in some embodiments, from about 5 kDa to about 150 kDa.

The present invention may be better understood with reference to the following examples.

### Test Methods

Melt Viscosity: The melt viscosity (Pa-s) may be determined in accordance with ISO Test No. 11443:2014 at a shear rate of 1,000 s⁻¹ and temperature 15° C. above the melting temperature using a Dynisco LCR7001 capillary rheometer. The rheometer orifice (die) had a diameter of 1 mm, length of 20 mm, L/D ratio of 20.1, and an entrance angle of 180°. The diameter of the barrel was 9.55 mm+0.005 mm and the length of the rod was 233.4 mm.

Melting Temperature: The melting temperature (“Tm”) may be determined by differential scanning calorimetry (“DSC”) as is known in the art. The melting temperature is the differential scanning calorimetry (DSC) peak melt temperature as determined by ISO Test No. 11357-2:2020. Under the DSC procedure, samples were heated and cooled at 20° C. per minute as stated in ISO Standard 10350 using DSC measurements conducted on a TA Q2000 Instrument.

Deflection Temperature Under Load (“DTUL”): The deflection under load temperature may be determined in accordance with ISO Test No. 75-2:2013 (technically equivalent to ASTM D648-18). More particularly, a test strip sample having a length of 80 mm, thickness of 10 mm, and width of 4 mm may be subjected to an edgewise three-point bending test in which the specified load (maximum outer fibers stress) was 1.8 Megapascals. The specimen may be lowered into a silicone oil bath where the temperature is raised at 2° C. per minute until it deflects 0.25 mm (0.32 mm for ISO Test No. 75-2:2013).

Tensile Modulus, Tensile Stress, and Tensile Elongation: Tensile properties may be tested according to ISO Test No. 527:2019 (technically equivalent to ASTM D638-14). Modulus and strength measurements may be made on the same test strip sample having a length of 80 mm, thickness of 10 mm, and width of 4 mm. The testing temperature may be 23° C., and the testing speeds may be 1 or 5 mm/min.

Flexural Modulus, Flexural Stress, and Flexural Elongation: Flexural properties may be tested according to ISO Test No. 178:2019 (technically equivalent to ASTM D790-10). This test may be performed on a 64 mm support span. Tests may be run on the center portions of uncut ISO 3167 multi-purpose bars. The testing temperature may be 23° C. and the testing speed may be 2 mm/min.

Charpy Impact Strength: Charpy properties may be tested according to ISO Test No. ISO 179-1:2010) (technically equivalent to ASTM D256-10, Method B). This test may be run using a Type 1 specimen size (length of 80 mm, width of 10 mm, and thickness of 4 mm). When testing the notched impact strength, the notch may be a Type A notch (0.25 mm base radius). Specimens may be cut from the center of a multi-purpose bar using a single tooth milling machine. The testing temperature may be 23° C.

### Example 1

A sample is formed that contained 85 wt. % LCP 1 and 15 wt. % glass fibers. LCP 1 is formed from about 73% HBA and 27% HNA.

### Example 2

A sample is formed that contained 85 wt. % LCP 2 and 15 wt. % glass fibers. LCP 2 is formed from about 79% HBA, 20% HNA, and 1% TA.

The samples noted above are injection molded into ISO tensile bars (80 mm×10 mm×4 mm) and tested for thermal and mechanical properties. The results are set forth below in Table 1.

TABLE 1

| | 1 | 2 |
|---|---|---|
| Melting Temperature | 280 | 321 |
| (° C., 1^{st} heat of DSC) | | |
| Melt Viscosity at 1,000 s⁻¹ (Pa-s) | 52.8 | 40.7 |
| Unnotched Charpy (kJ/m²) | — | 100 |
| Notched Charpy (kJ/m²) | 70 | 61 |
| Tensile Strength (MPa) | 200 | 154 |
| Tensile Modulus (MPa) | 12,000 | 12,600 |
| Tensile Elongation (%) | 3.1 | 2.0 |
| Flexural Strength (MPa) | 240 | 218 |
| Flexural Modulus (MPa) | 12,600 | 12,200 |
| DTUL (1.8 MPa, ° C.) | 230 | 242 |

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. A medical device for administering a drug compound to a user, wherein the medical device comprises a drug reservoir that includes the drug compound, a pump that is in fluid communication with the drug reservoir, and a housing that encloses the drug reservoir and the pump, wherein the medical device comprises a polymer composition containing a polymer matrix that includes a liquid crystalline polymer containing repeating units derived from naphthenic hydroxycarboxylic and/or dicarboxylic acids in an amount of about 25 mol. % or less of the polymer, wherein the polymer composition exhibits a melt viscosity of about 50 Pa-s or less as determined in accordance with ISO Test No. 11443:2014 at a shear rate of 1,000 seconds⁻¹ and temperature of about 30° C. above the melting temperature.

2. The medical device of claim 1, wherein the polymer composition exhibits a deflection temperature under load of from about 230° C. to about 300° C. as determined in accordance with ISO Test No. 75-2:2013 at a load of 1.8 Megapascals.

3. The medical device of claim 1, wherein the polymer composition exhibits a tensile elongation of from about 0.5% to about 2.5% as determined in accordance with ISO Test No. 527:2019 at a temperature of about 23° C.

4. The medical device of claim 1, wherein the polymer composition exhibits a tensile modulus of about 7,000 MPa or more as determined in accordance with ISO Test No. 527:2019 at a temperature of about 23° C.

5. The medical device of claim 1, wherein the liquid crystalline polymer contains repeating units derived from one or more aromatic dicarboxylic acids, one or more aromatic hydroxycarboxylic acids, or a combination thereof.

6. The medical device of claim 5, wherein the aromatic hydroxycarboxylic acids include 4-hydroxybenzoic acid, 6-hydroxy-2-naphthoic acid, or a combination thereof.

7. The medical device of claim 5, wherein the aromatic hydroxycarboxylic acids include terephthalic acid, isophthalic acid, 2,6-naphthalenedicarboxylic acid, or a combination thereof.

8. The medical device of claim 1, wherein the liquid crystalline polymer is wholly aromatic.

9. The medical device of claim 1, wherein the liquid crystalline polymer contains repeating units derived from naphthenic hydroxycarboxylic and/or dicarboxylic acids in an amount of from about 10 mol. % to about 25 mol. %.

10. The medical device of claim 1, wherein the liquid crystalline polymer contains repeating units derived from 6-hydroxy-2-naphthoic acid in an amount of about from about 10 mol. % to about 25 mol. %.

11. The medical device of claim 10, wherein the liquid crystalline polymer contains repeating units derived from 4-hydroxybenzoic acid in an amount of about from about 75 mol. % to about 90 mol. %.

12. The medical device of claim 1, wherein the polymer matrix constitutes from about 50 wt. % to about 98 wt. % of the polymer composition.

13. The medical device of claim 12, wherein the polymer composition further comprises a fibrous filler.

14. The medical device of claim 13, wherein the fibrous filler includes glass fibers.

15. The medical device of claim 13, wherein the fibrous filler constitutes from about 5 wt. % to about 25 wt. % of the polymer composition.

16. The medical device of claim 1, wherein the drug compound includes insulin.

17. The medical device of claim 1, wherein the housing comprises the polymer composition.

18. The medical device of claim 1, wherein the drug reservoir comprises the polymer composition.

19. The medical device of claim 1, wherein the pump comprises the polymer composition.

20. The medical device of claim 1, wherein the device further comprises an injection assembly that is configured to receive the drug compound from the pump and deliver the drug compound to the user.

21. The medical device of claim 20, wherein the injection assembly includes the polymer composition.

22. The medical device of claim 20, wherein the injection assembly includes a cannula that is sheathed by a sleeve.

23. The medical device of claim 22, wherein the cannula, sleeve, or a combination thereof include the polymer composition.
